(19) United States
(12) Patent Application Publication
(10) Pub. No.: US 2015/0297124 A1
(43) Pub. Date: Oct. 22, 2015

(54) OPTICAL BIOMETRIC DEVICE

(71) Applicant: SHIMADZU CORPORATION, Kyoto (JP)

(72) Inventor: Akihiro ISHIKAWA, Kyoto (JP)

(21) Appl. No.: 14/439,176

(22) PCT Filed: Nov. 8, 2012
(86) PCT No.: PCT/JP2012/078949

§ 371 Date: Apr. 28, 2015

(51) Int. Cl. A61B 5/1455; A61B 5/00

(52) U.S. Cl. CPC A61B 5/14551; A61B 5/6814; A61B 5/742; A61B 5/7278

(57) ABSTRACT

An optical biometric device includes a light sending/receiving unit having light sending probes placed on the head of a subject and light receiving probes placed on the head; a control unit for sending and receiving light to obtain M pieces of information about the amount of light received in M measurement portions; an operation unit for acquiring M pieces of measurement data based on the M pieces of information a display control unit for displaying N pieces of measurement data selected from among M pieces of measurement data on a display screen; and a process unit for processing at least one piece of measurement data selected from among the N measurement data. On the display screen, a measurement data image is selected so that measurement data to be processed in the process unit is determined, and the processed measurement data is displayed.

### TECHNICAL FIELD

The present invention relates to an optical biometric device for non-invasively measuring brain activity, and in particular, to an optical biometric device that can be used as an oxygen monitor or the like in order to diagnose whether or not tissue in the living body is normal by measuring chronological change in the blood flow or chronological change in oxygen supply in each portion within the brain.

### BACKGROUND ART

In recent years, optical imaging devices for simply and non-invasively measuring brain functions using light have been developed in order to observe the state of brain activity. In these optical imaging devices for measuring the brain functions, light sending probes placed on the surface of the head of a subject irradiate the brain with near-infrared rays having three different wavelengths: λ₁, λ₂ and λ₃ (780 nm, 805 nm and 830 nm, for example), and at the same time, light receiving probes placed on the surface of the head detect changes in the intensity of the near-infrared rays (information about the amount of received light) ΔA(λ₁), ΔA(λ₂) and ΔA(λ₃) of the respective wavelengths λ₁, λ₂ and λ₃ emitted from the brain.

In order to find the product of the change in the concentration of the oxyhemoglobin in the blood flow in the brain and the length of the optical path [oxyHb] and the product of the change in the concentration of the deoxyhemoglobin and the length of the optical path [deoxyHb] from the thus-obtained information on the amounts of received light ΔA(λ₁), ΔA(λ₂) and ΔA(λ₃), simultaneous equations (1) to (3) are created using the modified Beer-Lambert Law, for example, and the simultaneous equations are solved. Furthermore, the product of the change in the concentration of the total amount of hemoglobin and the length of the optical path ([oxyHb]+[deoxyHb]) is calculated from the product of the change in the concentration of oxyhemoglobin and the length of the optical path [oxyHb] and the product of the change in the concentration of deoxyhemoglobin and the length of the optical path [deoxyHb].

ΔA(λ₁)=Eₒ(λ₁)×[oxyHb]+E_{d}(λ₁)×[deoxyHb] (1)

ΔA(λ₂)=Eₒ(λ₂)×[oxyHb]+E_{d}(λ₂)×[deoxyHb] (2)

ΔA(λ₃)=Eₒ(λ₃)×[oxyHb]+E_{d}(λ₃)×[deoxyHb] (3)

Here, Eₒ(λₘ) is the absorbance coefficient of oxyhemoglobin for light having a wavelength λₘ, and E_{d}(λₘ) is the absorbance coefficient of deoxyhemoglobin for light having a wavelength λₘ.

In addition, a near-infrared spectrometer or the like is used in the optical imaging devices for measuring the brain functions in order to measure the product of the change in the concentration of oxyhemoglobin and the length of the optical path [oxyHb], the product of the change in the concentration of deoxyhemoglobin and the length of the optical path [deoxyHb], and the product of the change in the concentration of the total amount of hemoglobin and the length of the optical path ([oxyHb]+[deoxyHb]), respectively, in a number of measurement portions in the brain (see Patent Document 1).

In such a near-infrared spectrometer a holder (light sending/receiving unit) is used in order to make 15 light sending probes and 15 light receiving probes make contact on the surface of the head of a patient in a predetermined arrangement. FIG. 2 is a plan diagram showing an example of a holder into which 15 light sending probes and 15 light receiving probes have been inserted.

Light sending probes 12_{T1} through 12_{T15} and light receiving probes 13_{R1} through 13_{R15} are aligned alternately in a matrix of five probes in the longitudinal direction and six probes in the lateral direction. At this time the intervals between the light sending probes 12_{T1} through 12_{T15} and light receiving probes 13_{R1} through 13_{R15} are 30 mm As a result, information about the amount of light received in 49 measurement portions in the brain ΔAₙ(λ₁), ΔAₙ(λ₂) and ΔAₙ(λ₃) (n=1, 2 . . . , 49) is obtained.

Thus, 49 pieces of information about the amount of received light are gained within a predetermined interval of time Δt so that chronological change (measurement data) Xₙ(t) in the product of the change in the concentration of oxyhemoglobin and the length of the optical path [oxyHb], chronological change (measurement data) Yₙ(t) in the product of the change in the concentration of deoxyhemoglobin and the length of the optical path [deoxyHb] and chronological change (measurement data) Zₙ(t) (n=1, 2 . . . , 49) in the product of the change in the concentration of the total amount of hemoglobin and the length of the optical path ([oxyHb]+[deoxyHb]) can be found using equations (1), (2) and (3), and displayed.

FIG. 7 is a diagram showing a display screen on which pieces of measurement data Xₙ(t), Yₙ(t) and Zₙ(t) for 49 measurement portions are aligned. The longitudinal axis shows the product of the change in the concentration and the length of the optical path [oxyHb], and the lateral axis shows the time t of the measurement data. In addition, the channel number n (n=1, 2 . . . , 49) shows the relationship between a light sending probe 12 and a light receiving probe 13 from which the measurement data has been gained, and is displayed in the upper left portion of each piece of measurement data.

Thus, 49 pieces of measurement data Xₙ(t), Yₙ(t) and Zₙ(t) are displayed. In the plan diagram in FIG. 2, pieces of measurement data are displayed in a matrix of light sending probes 12_{T1} through 12_{T15} and light receiving probes 13_{R1} through 13_{R15} in such a manner that each piece of measurement data, gained when light emitted from a light transmitting probe 12 is detected by a light receiving probe 13, is located at the midpoint of the line segment connecting the light sending probe 12 and the light receiving probe 13. Concretely, 49 measurement images #1 through #49 are aligned in such a manner that the piece of measurement data gained when light emitted from the light sending probe 12_{T1} is detected by the light receiving probe 13_{R1} is located in the upper left as a measurement data image #1 with a channel number 1, the piece of measurement data gained when light emitted from the light sending probe 12_{T2} is detected by the light receiving probe 13_{R1} is located on the right of the measurement data image #1 as a measurement data image #2 with a channel number 2, and the piece of measurement data gained when light emitted from the light sending probe 12_{T1} is detected by the light receiving probe 13_{R4} is located to the lower left of the measurement data image #1 as a measurement data image #6 with a channel number 6.

As shown in FIG. 7, a signal based on a change in epidermal blood flow, fluctuation in the heart rate or a change in the pulse or respiration is superposed on the displayed 49 pieces of measurement data #1 through #49, in addition to a signal based on blood flow resulting from brain activation.

Thus, various processes are carried out on the measurement data #1 through #49 so that whether or not symptoms indicating brain ischemia or the like are present can be easily diagnosed. For example, an addition process for adding up four pieces of measurement data selected from the 49 pieces of measurement data #1 through #49, a statistical process for calculating statistical data from 38 pieces of measurement data selected from the 49 pieces of measurement data #1 through #49, a display enlargement process for enlarging a display of four pieces of measurement data selected from the 49 pieces of measurement data #1 through #49, and a data output process for displaying four pieces of measurement data selected from the 49 pieces of measurement data #1 through #49 in a numerical value table are carried out.

FIG. 8 is a diagram showing an input screen for processing the 49 pieces of measurement data #1 through #49. The input screen displays a box in which channel numbers for the pieces of measurement data, on which an addition process is desired to be carried out, are inputted, a box in which channel numbers for the pieces of measurement data, on which a statistical process is desired to be carried out, are inputted, a box in which channel numbers for the pieces of measurement data, on which a display enlargement process is desired to be carried out, are inputted, and a box in which channel numbers for the pieces of measurement data, on which a data output process is desired to be carried out, are inputted. In addition, an “OK” button and an “information clear” button are displayed at the bottom of the input screen.

As a result, according to the prior art, doctors, or the like, observe the display screen as in FIG. 7 and record channel numbers of the pieces of measurement data that are desired to be processed in a notebook or the like and, then, call up an input screen as in FIG. 8, into which the channel numbers of the pieces of measurement data are inputted into boxes on the input screen before clicking the “OK” button.

### PRIOR ART DOCUMENT

### Patent Document

Patent Document 1: Japanese Unexamined Patent Publication 2006-109964

### SUMMARY OF THE INVENTION

### Problem to Be Solved by the Invention

When using a conventional near infrared spectrometer it is necessary to switch between screens because the channel numbers of the pieces of measurement data that are desired to be processed have been inputted to a screen that is different from the screen for displaying the measurement data #1 through #49. Therefore, a wrong channel number may be recorded in a notebook or the like, or a wrong channel number may be inputted when there is a failure to recognize the positional relationship between the channel numbers.

Thus, an object of the present invention is to provide an optical biometric device with which measurement data can be easily processed while the measurement data is being observed.

### Means for Solving Problem

In order to solve the above described problem, the optical biometric device according to the present invention is provided with: a light sending/receiving unit having a number of light sending probes to be placed on a surface of the head of a subject and a number of light receiving probes to be placed on a surface of the head; a control unit for sending and receiving light which acquires M pieces of information about the amount of light received in M measurement portions under control such that the above described light sending probes irradiate the surface of the head with light, and at the same time the above described light receiving probes detect light emitted from the surface of the head; an operation unit for acquiring M pieces of measurement data on the basis of the M pieces of information about the amount of received light; a measurement data display control unit for displaying a display screen on which N pieces of measurement data selected from among M pieces of measurement data are aligned; and a process unit for processing at least one piece of measurement data selected from among the N measurement data, and is characterized in that, on a display screen displayed by the above described measurement data display control unit, a measurement data image is selected so that measurement data to be processed in the above described process unit is determined, and the processed measurement data is displayed.

Here, “measurement data” may be chronological change itself in the information about the amount of received light that has been detected by a light receiving probe, chronological change in the concentration of oxyhemoglobin that has been calculated from the information about the amount of received light, chronological change in the concentration of deoxyhemoglobin that has been calculated from the information about the amount of received light, chronological change in the concentration of the total amount of hemoglobin that has been calculated from the information about the amount of received light, information itself about the amount of received light at a certain point in time, the concentration of oxyhemoglobin at a certain point in time, the concentration of deoxyhemoglobin at a certain point in time or the concentration of the total amount of hemoglobin at a certain point in time.

In the optical biometric device according to the present invention, the measurement data display control unit displays a display screen on which N pieces of measurement data are aligned. Thus, a doctor, or the like, observes the display screen and selects the pieces of measurement data that are desired to be processed on the display screen. Accordingly, it is not necessary for the doctor, or the like, to memorize the channel numbers of the pieces of measurement data that are desired to be processed and, thus, it is not necessary to carry out a switching operation for the opening of another screen as in the prior art.

### Effects of the Invention

With the optical biometric device according to the present invention, measurement data can be easily processed while observing the measurement data. At this time, the doctor, or the like, can take into consideration the positional relationship between pieces of measurement data and the quality of the measurement data and, in addition, can prevent mistaken operation, such as an error in selection.

(Other Means for Solving Problem and Effects Thereof)

Alternatively, the optical biometric device according to the present invention is provided with: a light sending/receiving unit having a number of light sending probes to be placed on a surface of the head of a subject and a number of light receiving probes to be placed on a surface of the head; a control unit for sending and receiving light which acquires M pieces of information about the amount of light received in M measurement portions under control such that the above described light sending probes irradiate the surface of the head with light, and at the same time the above described light receiving probes detect light emitted from the surface of the head; an operation unit for acquiring M pieces of measurement data on the basis of the M pieces of information about the amount of received light; a measurement data display control unit for displaying a display screen on which N pieces of measurement data selected from among M pieces of measurement data are aligned; and a process unit for processing at least one piece of measurement data selected from among the N measurement data, and is characterized in that the above described measurement data display control unit displays a display screen on which N measurement data buttons corresponding to the locations of the aligned N pieces of measurement data are aligned, and on a display screen displayed by the above described measurement data display control unit, a measurement data button is selected so that measurement data to be processed in the above described process unit is determined, and the processed measurement data is displayed.

As described above, with the optical biometric device according to the present invention, measurement data can be easily processed. At this time, the doctor, or the like, can take into consideration the positional relationship between pieces of measurement data and the quality of the measurement data and, in addition, can prevent mistaken operation, such as an error in selection.

In addition, in the optical biometric device according to the present invention the method for displaying a selected measurement data image or measurement data button may be able to be changed by selecting a measurement data image or a measurement data button on the display screen displayed in the above described measurement data display control unit.

The optical biometric device according to the present invention can aid the user in the selection of the measurement data.

Furthermore, in the optical biometric device according to the present invention, the above described display method may consist of the change in the color of the above described measurement data image or the above described measurement data button, or the addition of a mark to the above described measurement data image or the above described measurement data button.

Thus, in the optical biometric device according to the present invention, a number of measurement data images or a number of measurement data buttons may be selected on the display screen displayed in the above described measurement data display control unit before the above described process unit processes the selected pieces of measurement data.

Moreover, in the optical biometric device according to the present invention, the above described process unit may carry out at least one process selected from a group of processes that consists of an addition process for adding a number of pieces of measurement data, a statistical process for calculating statistical data from a number of pieces of measurement data, a display enlargement process for enlarging the display of measurement data, and a data output process for displaying measurement data in a numerical value table.

### BRIEF DESCRIPTION OF THE DRAWINGS

FIG. 1 is a block diagram schematically showing the structure of the optical biometric device according to one embodiment of the present invention;

FIG. 2 is a plan diagram showing an example of a holder into which 15 light sending probes and 15 light receiving probes are inserted;

FIG. 3 is a diagram showing a display screen on which pieces of measurement data in 49 measurement portions are aligned;

FIG. 4 is a diagram showing a display screen where 38 pieces of measurement data are selected;

FIG. 5 is a diagram showing a display screen where measurement data Xₙ(t) in 49 measurement portions is expressed as a contour map and four pieces of measurement data are selected;

FIG. 6 is diagram showing a display screen where 49 measurement data buttons are aligned and four measurement data buttons are selected;

FIG. 7 is diagram showing a display screen where pieces of measurement data in 49 measurement places are aligned; and

FIG. 8 is a diagram showing an input screen for processing 49 pieces of measurement data #1 through #49.

### PREFERRED EMBODIMENT OF THE INVENTION

In the following, the preferred embodiments of the present invention are described in reference to the drawings. Here, the present invention is not limited to the below described embodiments but includes various modifications as long as the gist of the present invention is not deviated from.

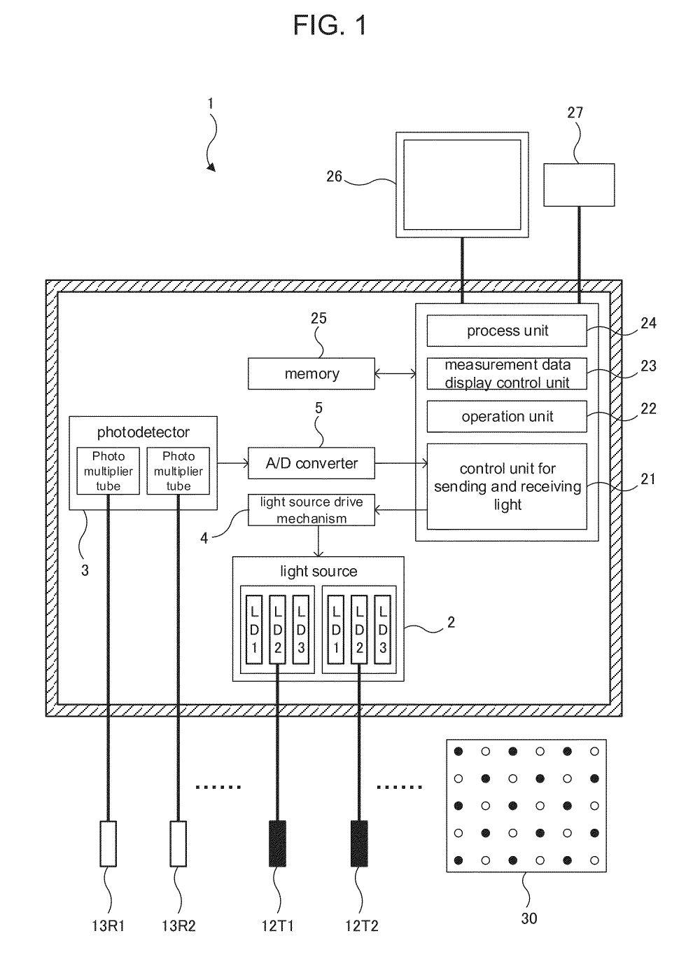

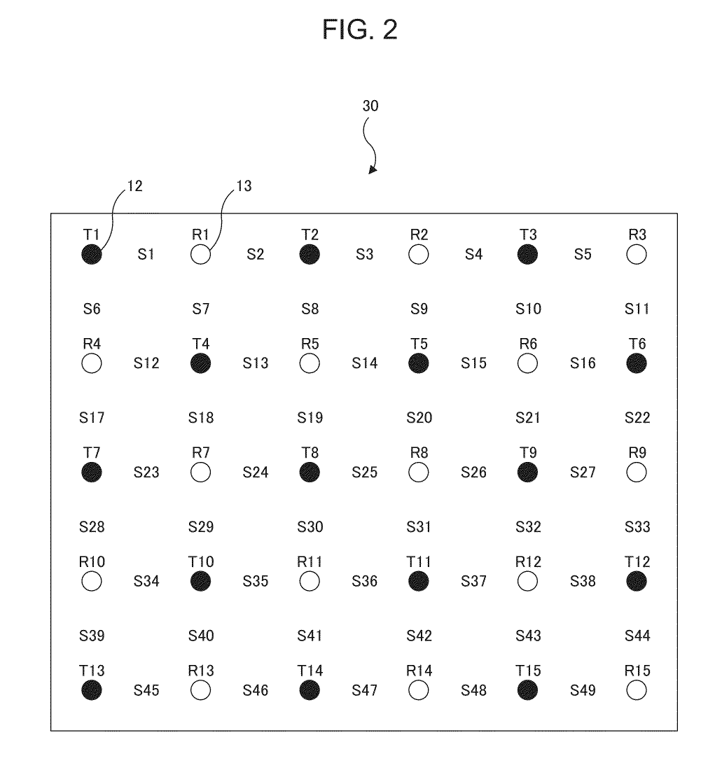

FIG. 1 is a block diagram schematically showing the structure of the optical biometric device according to one embodiment of the present invention. An optical biometric device 1 is provided with a light source 2 for emitting light, a light source drive mechanism for driving the light source 2, a photodetector 3 for detecting light, an A/D converter 5, a control unit 21 for sending and receiving light, an operation unit 22, a measurement data display control unit 23, a process unit 24, a memory 25, 15 light sending probes 12, 15 light receiving probes 13, a holder 30, a display unit 26 and a keyboard 27.

The light source drive mechanism 4 drives the light source 2 in response to a drive signal inputted from the control unit 21 for sending and receiving light. The light source 2 includes semiconductor lasers LD1, LD2, and LD3 that can emit near infrared rays having three different wavelengths λ₁, λ₂ and λ₃, for example.

The photodetector 3 is a photomultiplier tube or the like and individually detects near infrared rays received by the 15 light receiving probes 13_{R1} through 13_{R15} so that 15 pieces of information about the amount of received light ΔAₙ(λ₁), ΔAₙ(λ₂) and ΔAₙ(λ₃) are outputted to the control unit 21 for sending and receiving light via the A/D converter 5.

15 light sending probes 12_{T1} through 12_{T15} and 15 light receiving probes 13_{R1} through 13_{R15} are inserted into the holder 30. The light sending probes 12_{T1} through 12_{T15} and the light receiving probes 13_{R1} through 13_{R15} are alternately aligned in both the row and column directions in a tetragonal lattice form. Here, the intervals between the light sending probes 12_{T1} through 12_{T15} and the light receiving probes 13_{R1} through 13_{R15} are 30 mm.

The control unit 21 for sending and receiving light outputs a drive signal, which sends light to one light probe 12, to the light source drive mechanism 4 at a predetermined time and, at the same time, carries out control for allowing the photodetector 3 to detect information about the amount of light received by the light receiving probes 13 ΔAₙ(λ₁), ΔAₙ(λ₂) and ΔAₙ(λ₃) (n=1, 2 . . . , 49). Specifically, light is sent sequentially to one light sending probe each, 12_{T1} through 12_{T15}, in accordance with a predetermined timing wherein during the first 5 milliseconds light having a wavelength of 780 nm is sent to the light sending probe 12_{T1}, during the next 5 milliseconds light having a wavelength of 805 nm is sent to the light sending probe 12_{T1}, during the next 5 milliseconds light having a wavelength of 830 nm is sent to the light sending probe 12_{T1}, and during the next 5 milliseconds light having a wavelength of 780 nm is sent to the light sending probe 12_{T1}. At this time the 15 light receiving probes 13_{R1} through 13_{R15} detect information about the amount of received light whenever light is sent to any one of the light sending probes 12_{T1} through 12_{T15,} and the memory 25 stores the information about the amount of light detected by a predetermined light receiving probe from among the light receiving probes 13_{R1} through 13_{R15} (adjacent to the light sending probe that has emitted light) in accordance with a predetermined timing. As a result, 49 pieces of information in total about the amount of received light ΔAₙ(λ₁), ΔAₙ(λ₂) and ΔAₙ(λ₃) are collected.

The operation unit 22 carries out control for finding chronological change (measurement data) Xₙ(t)in the product of the change in the concentration of oxyhemoglobin and the length of the optical path [oxyHb], chronological change (measurement data) Yₙ(t)in the product of the change in the concentration of deoxyhemoglobin and the length of the optical path [deoxyHb] and chronological change (measurement data) Zₙ(t)(n=1, 2 . . . , 49) in the product of the change in the concentration of the total amount of hemoglobin and the length of the optical path ([oxyHb]+[deoxyHb]) on the basis of the 49 pieces of information about the amount of received light ΔAₙ(λ₁), ΔAₙ(λ₂) and ΔAₙ(λ₃) stored in the memory 25 using equations (1), (2), and (3).

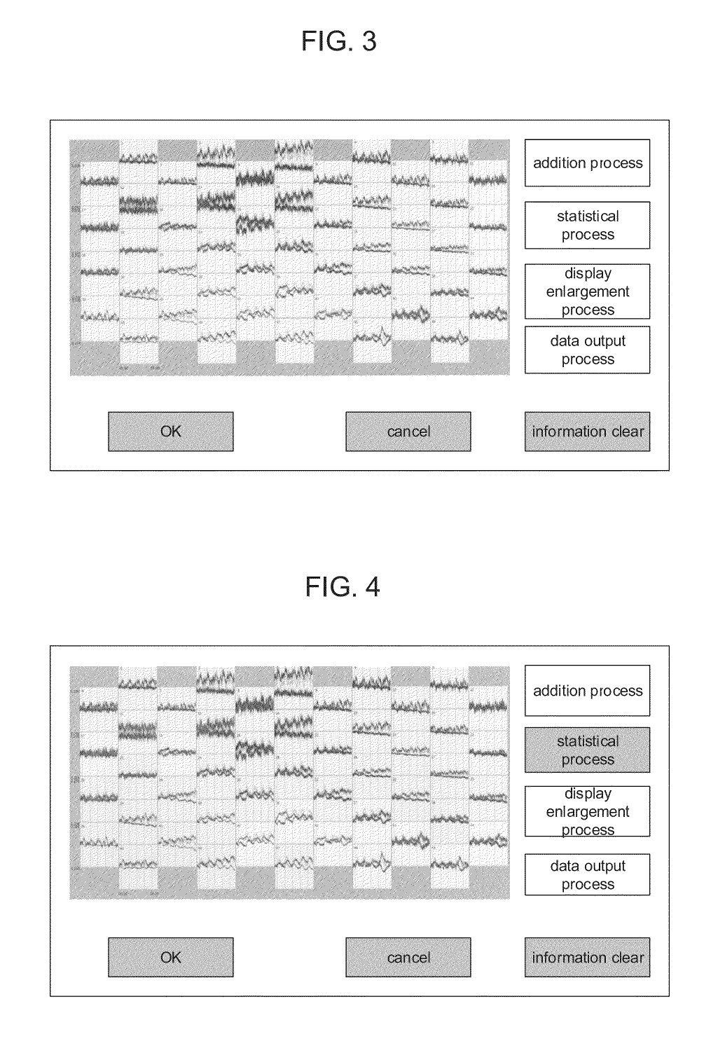

The measurement data display control unit 23 carries out control for displaying measurement data #1 through #49 calculated by the operation unit 22. FIG. 3 is a diagram showing a display screen on which pieces of measurement data Xₙ(t), Yₙ(t) and Zₙ(t) in 49 measurement portions are aligned.

49 measurement data images #1 through #49 are aligned and displayed on the display screen. In addition, the channel number n (n=1, 2 . . . , 49) that indicates the relationship between the light sending probe 12 and the light receiving probe 13 according to which the measurement data is gained is displayed on the upper left of each measurement data image #1 through #49. In addition, the color of the background of the measurement data images #1 through #49 is white. An “OK” button, a “cancel” button and an “information clear” button are displayed at the bottom of the display screen, and the usage method is described below. In addition, an “addition process” button, a “statistical process” button, a “display enlargement process” button and a “data output process” button are displayed on the right of the display screen.

The process unit 24 carries out control for processing and displaying the selected measurement data by selecting a measurement data image from among the 49 measurement data images #1 through #49 on the display screen in FIG. 3. FIG. 4 is a diagram showing a display screen where 38 pieces of measurement data #1, #6, #7, and #12 are selected.

It is assumed, for example, that a doctor, or the like, would observe the display screen in FIG. 3 and would want to carry out a statistical process for calculating statistical data from 38 pieces of measurement data excluding 11 pieces of measurement data #39, #40, #41, #42, #43, #44, #45, #46, #47, #48, and #49. At this time, the “statistical process” button is touched with a finger or a touch pen and the measurement data screen #1 is touched so that the color of the background of the measurement data image #1 turns to gray, the measurement data screen #2 is touched so that the color of the background of the measurement data image #2 turns to gray and, in the same manner, the 38 measurement data images are touched so that the color of the background of the 38 measurement data images turns to gray (see FIG. 4). Then, the “OK” button is touched. As a result, the process unit 24 calculates statistical data from the 38 pieces of measurement data excluding the 11 pieces of measurement data #39, #40, #41, #42, #43, #44, #45, #46, #47, #48, and #49, and displays the statistical data.

In the case wherein the measurement data image #39 is mistakenly selected, the “cancel” button is touched and the measurement data image #39 is touched so that the color of the background of the measurement data image #39 turns to white and, thus, the selection of the measurement data image #39 is canceled. In the case wherein it becomes unnecessary to carry out a statistical process after the measurement data image #7 is touched, for example, the “information clear” button is touched so that the color of the background of the measurement data images #1 through #7 turns to white and all of the selections are cancelled.

In addition, it is assumed that a doctor, or the like, would observe the display screen in FIG. 3 and would want to carry out an addition process for adding up the measurement data #1, the measurement data #6, the measurement data #7, and the measurement data #12. At this time, the “addition process” button is touched and the measurement data image #1 is touched so that the color of the background of the measurement data image #1 is turned to gray, the measurement data image #6 is touched so that the color of the background of the measurement data image #6 is turned to gray, the measurement data image #7 is touched so that the color of the background of the measurement data image #7 is turned to gray, the measurement data image #12 is touched so that the color of the background of the measurement data image #12 is turned to gray and, then, the “OK” button is touched. As a result, the process unit 24 displays added measurement data gained by carrying out an addition process on the measurement data #1, the measurement data #6, the measurement data #7, and the measurement data #12.

Furthermore, it is assumed that a doctor, or the like, would observe the display screen in FIG. 3 and would want to carry out a display enlargement process for enlarging the display of the measurement data #1, the measurement data #6, the measurement data #7, and the measurement data #12. At this time, the “display enlargement process” button is touched and the measurement data image #1 is touched so that the color of the background of the measurement data image #1 is turned to gray, the measurement data image #6 is touched so that the color of the background of the measurement data image #6 is turned to gray, the measurement data image #7 is touched so that the color of the background of the measurement data image #7 is turned to gray, the measurement data image #12 is touched so that the color of the background of the measurement data image #12 is turned to gray and, then, the “OK” button is touched. As a result, the process unit 24 displays the enlarged measurement data gained by carrying out a display enlargement process on the measurement data #1, the measurement data #6, the measurement data #7, and the measurement data #12.

Moreover, it is assumed that a doctor, or the like, would observe the display screen in FIG. 3 and would want to carry out a data output process for displaying the measurement data #1, the measurement data #6, the measurement data #7, and the measurement data #12 in a numerical value table. At this time, the “data output process” button is touched and the measurement data image #1 is touched so that the color of the background of the measurement data image #1 is turned to gray, the measurement data image #6 is touched so that the color of the background of the measurement data image #6 is turned to gray, the measurement data image #7 is touched so that the color of the background of the measurement data image #7 is turned to gray, the measurement data image #12 is touched so that the color of the background of the measurement data image #12 is turned to gray and, then, the “OK” button is touched. As a result, the process unit 24 displays the measurement data #1, the measurement data #6, the measurement data #7, and the measurement data #12 in a numerical value table.

As described above, with the optical biometric device 1 measurement data can be easily processed while 49 pieces of measurement data #1 through #49 are being observed. At this time, the doctor, or the like, can take into consideration the positional relationship between pieces of measurement data and the quality of the measurement data and, in addition, can prevent mistaken operation, such as an error in selection.

### Other Embodiments

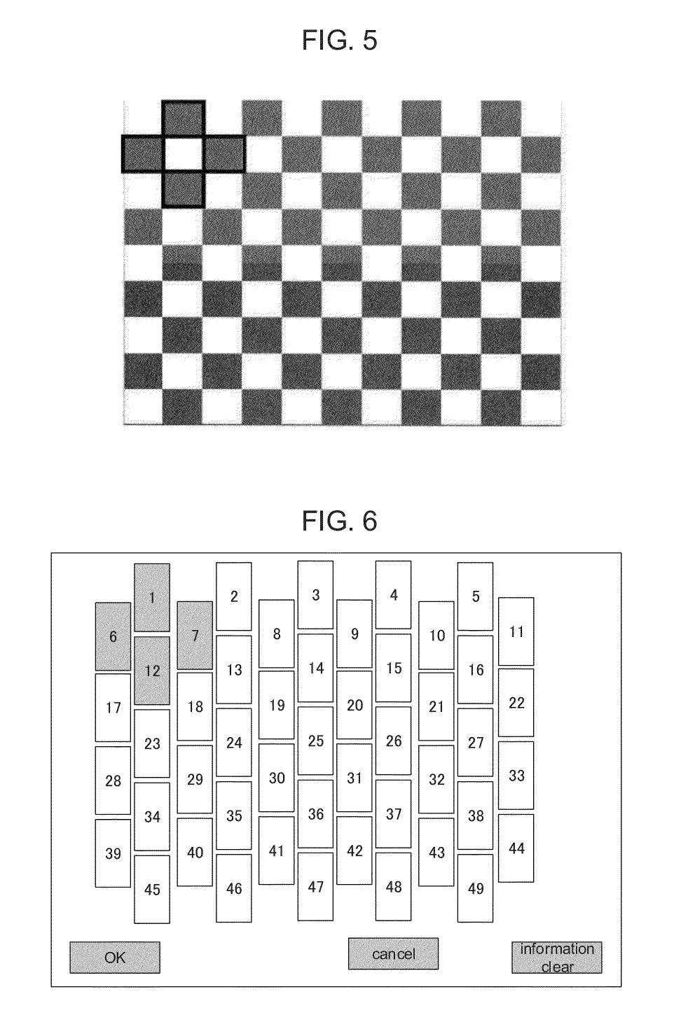

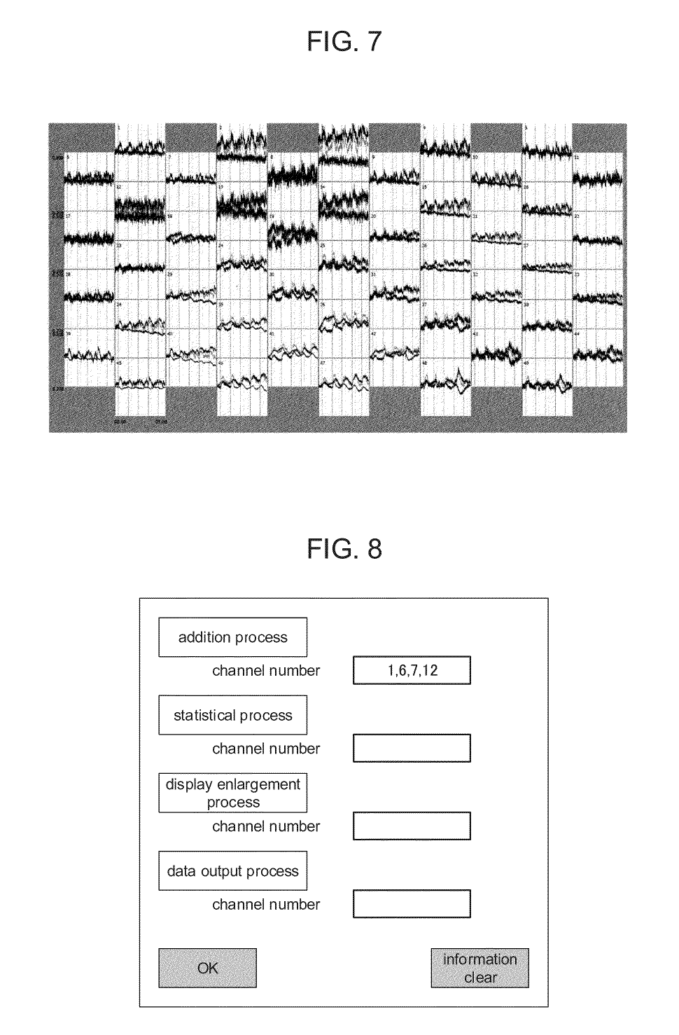

(1) Though the above described optical biometric device 1 has a configuration wherein the measurement data display control unit 23 shows chronological change (measurement data) Xₙ(t) in the product of the change in the concentration of oxyhemoglobin and the length of the optical path [oxyHb], chronological change (measurement data) Yₙ(t) in the product of the change in the concentration of deoxyhemoglobin and the length of the optical path [deoxyHb] and chronological change (measurement data) Zₙ(t) (n=1, 2 . . . , 49) in the product of the change in the concentration of the total amount of hemoglobin and the length of the optical path ([oxyHb]+[deoxyHb]) as measurement data #1 through #49, the configuration may allow the product of the change in the concentration of oxyhemoglobin and the length of the optical path [oxyHb] at a certain point in time to be expressed by color as measurement data. FIG. 5 is a diagram showing a display screen where the measurement data Xₙ(t) in 49 measurement portions is expressed as a contour map, and four pieces of measurement data #1, #6, #7, and #12 are selected.

(2) Though the above described optical biometric device 1 has a configuration wherein measurement data images are selected from among 49 measurement data images #1 through #49 on the display screen in FIG. 3, the configuration may allow a display screen wherein 49 measurement data buttons are aligned so as to correspond to the locations of the 49 pieces of measurement data that are aligned to be displayed so that measurement data buttons can be selected from among the 49 measurement data buttons. FIG. 6 is a diagram showing a display screen wherein 49 measurement data buttons are aligned and four measurement data buttons are selected.

(3) Though the above described optical biometric device 1 has a configuration wherein measurement data images are touched with a finger or a touch pen, the configuration may allow measurement data images to be clicked, double clicked, or pressed down for relatively long period of time using a scroll cursor.

(4) Though the above described optical biometric device 1 has a configuration wherein the color of the background of measurement data images changes, the configuration may allow the color of the channel number n displayed on the measurement data images to change or may allow the channel number n to be highlighted.

(5) Though the above described optical biometric device 1 has a configuration wherein a holder 30 having 15 light sending probes 12_{T1} through 12_{T15} and 15 light receiving probes 13_{R1} through 13_{R15} is used, the configuration may allow a holder having eight light sending probes 12_{T1} through 12_{T8} and eight light receiving probes 13_{R1} through 13_{R8} to be used.

### INDUSTRIAL APPLICABILITY

The present invention can be applied to an optical biometric device for non-invasively measuring brain activity.

### EXPLANATION OF SYMBOLS

1: optical biometric device

12: light sending probes

13: light receiving probes

21: control unit for sending and receiving light

22: operation unit

23: measurement data display control unit

24: operation unit

30: holder (light sending/receiving unit)

1. An optical biometric device, comprising:
a light sending/receiving unit having a number of light sending probes to be placed on a surface of the head of a subject and a number of light receiving probes to be placed on a surface of the head;
a control unit for sending and receiving light which acquires M pieces of information about the amount of light received in M measurement portions under control such that said light sending probes irradiate the surface of the head with light, and at the same time said light receiving probes detect light emitted from the surface of the head;
an operation unit for acquiring M pieces of measurement data on the basis of the M pieces of information about the amount of received light;
a measurement data display control unit for displaying a display screen on which N pieces of measurement data selected from among M pieces of measurement data are aligned; and
a process unit for processing at least one piece of measurement data selected from among the N measurement data, characterized in that
on a display screen displayed by said measurement data display control unit, a measurement data image is selected so that measurement data to be processed in said process unit is determined, and the processed measurement data is displayed.

2. An optical biometric device, comprising:
a light sending/receiving unit having a number of light sending probes to be placed on a surface of the head of a subject and a number of light receiving probes to be placed on a surface of the head;
a control unit for sending and receiving light which acquires M pieces of information about the amount of light received in M measurement portions under control such that said light sending probes irradiate the surface of the head with light, and at the same time said light receiving probes detect light emitted from the surface of the head;
an operation unit for acquiring M pieces of measurement data on the basis of the M pieces of information about the amount of received light;
a measurement data display control unit for displaying a display screen on which N pieces of measurement data selected from among M pieces of measurement data are aligned; and
a process unit for processing at least one piece of measurement data selected from among the N measurement data, characterized in that
said measurement data display control unit displays a display screen on which N measurement data buttons corresponding to the locations of the aligned N pieces of measurement data are aligned, and
on a display screen displayed by said measurement data display control unit, a measurement data button is selected so that measurement data to be processed in said process unit is determined, and the processed measurement data is displayed.

3. The optical biometric device according to claim 1, characterized in that the method for displaying the selected measurement data image or measurement data button is changed by selecting a measurement data image or a measurement data button on a display screen displayed in said measurement data display control unit.

4. The optical biometric device according to claim 3, characterized in that said method allows for changing of the color of the measurement data image or of the measurement data button, or for adding a mark to the measurement data image or to the measurement data button.

5. The optical biometric device according to claim 1, characterized in that said process unit processes the selected pieces of measurement data after a number of measurement data images or measurement data buttons have been selected on a display screen displayed in said measurement data display control unit.

6. The optical biometric device according to claim 1, characterized in that said process unit carries out at least one process selected from the process group consisting of an addition process for adding a number of pieces of measurement data, a statistical process for calculating statistical data from a number of pieces of measurement data, a display enlargement process for enlarging the display of measurement data, and a data output process for displaying measurement data in a numerical value table.
